(19) United States
(12) Patent Application Publication
(10) Pub. No.: US 2024/0408142 A1
(43) Pub. Date: Dec. 12, 2024

(54) PREPARATION METHOD FOR CANINE WHOLE BLOOD HEMATOPOIETIC STEM CELLS

(71) Applicant: Qingdao Jinmotang Biotechnology Co., LTD., Qingdao (CN)

(72) Inventors: Feng SHAO, Qingdao (CN); Shuai GAO, Qingdao (CN); Juan WANG, Qingdao (CN); Chunyu SHAO, Qingdao (CN)

(73) Assignee: Qingdao Jinmotang Biotechnology Co., LTD., Qingdao (CN)

(21) Appl. No.: 18/206,091

(22) Filed: Jun. 6, 2023

(51) Int. Cl. A61K 35/28

(52) U.S. Cl. CPC A61K 35/28

(57) ABSTRACT

A preparation method for canine whole blood hematopoietic stem cells includes the following steps: step 1: performing matching of specific pathogen free (SPF) dogs, where the blood type of the SPF dogs is negative blood, the SPF dogs need to be immunized with a conventional pentavaccine, an indirect enzyme-linked immunosorbent assay (ELISA) method is used for testing, the antibody titer thereof reaches over 2⁹, and the SPF dogs need to be matched with diseased dogs; and step 2: performing physical examination of the dogs, where venous blood is regularly collected for hematology and serum biochemical testing before blood sampling of the matched dogs. According to the present disclosure, the experimental dogs, namely the SPF dogs are free of special pathogens, and have great advantages in the aspect of safety performance, such that the prepared blood has good safety performance.

### TECHNICAL FIELD

The present disclosure relates to the field of blood products, and in particular to a preparation method for canine whole blood hematopoietic stem cells.

### BACKGROUND

Hematopoietic stem cell transplantation has been developed for about half a century, and is currently widely used in treatment of blood system diseases, and more than one hundred thousand patients in the global range accept various hematopoietic stem cell transplantation. According to a source of the donor, the hematopoietic stem cell transplantation can be divided into: autologous hematopoietic stem cell transplantation (Auto-SCT), allogeneic hematopoietic stem cell transplantation (between identical twin brothers or sisters), and heterogenic hematopoietic stem cell transplantation (Allo-SCT). Treatment of malignant diseases by means of the hematopoietic stem cell transplantation accounts for the majority of clinical cases. In the present disclosure, a method for obtaining hematopoietic stem cells from peripheral blood by using specific pathogen free (SPF) dogs is employed to provide perfect hematopoietic stem cells for diseased dogs.

### SUMMARY

An objective of the present disclosure is to provide a preparation method for canine whole blood hematopoietic stem cells so as to solve the problems put forward in the back art.

To achieve the above objective, the present disclosure employs the following technical solution:

A preparation method for canine whole blood hematopoietic stem cells includes the following steps:
step 1: performing matching of dogs, where the blood type of the dogs is negative blood, the dogs need to be immunized with a conventional pentavaccine, an indirect enzyme-linked immunosorbent assay (ELISA) method is used for testing, the antibody titer thereof reaches over 2⁹, and the dogs need to be matched with diseased dogs;
step 2: performing physical examination of the dogs, where venous blood is regularly collected for hematology and serum biochemical testing before blood sampling of the matched dogs, and each index is within a normal range; and
step 3: collecting hematopoietic stem cells of the dogs, where a mobilization agent is injected every day before the hematopoietic stem cells are collected, and the dogs need to be fed more royal jelly before the hematopoietic stem cells are collected so as to improve a content of the hematopoietic stem cells.

Preferably, step 1, step 2 and step 3 are all performed in a complete sterile closed room.

Preferably, the dogs are specific pathogen free (SPF) dogs, and the SPF dogs include greyhounds, Beagle dogs, Dobermans, boxer dogs, and German shepherd dogs, and the SPF dogs have an age of 2-4 years old and a weight of 20-40 kg.

Preferably, the mobilization agent is a recombinant human granulocyte colony stimulating factor injection: rhG-CSF.

Preferably, the injection amount of the mobilization agent is 150 μg/kg per day, and subcutaneous injection is performed once per day for five continuous days.

Preferably, first time of hematopoietic stem cell collection is performed on the fifth day of injection of the mobilization agent.

Preferably, when the hematopoietic stem cells collected for the first time are not enough, second time of collection is performed.

Compared with the prior art, the present disclosure provides the preparation method for canine whole blood hematopoietic stem cells. The method has the following beneficial effects:

According to the present disclosure, the experimental dogs, namely the SPF dogs are free of special pathogens, and have great advantages in the aspect of safety performance, such that the prepared blood has good safety performance. The hematopoietic stem cells are provided for other diseased dogs by using SPF greyhounds as volunteers, which not only improves the safety of blood transfusion, but also greatly improves a cure rate of the diseased dogs, and plays a crucial role in diagnosis and treatment of the diseased dogs.

### DETAILED DESCRIPTION OF THE EMBODIMENTS

The technical solutions in the examples of the present disclosure will be clearly and completely described below. Apparently, the described examples are merely some examples rather than all examples of the present disclosure.

### Example 1

Greyhounds aged at 2-4 years old and having negative blood and a weight of 20-40 kg were selected.

The dogs were immunized with a conventional pentavaccine, an indirect enzyme-linked immunosorbent assay (ELISA) method was used for testing, and the antibody titer thereof reaches over 2⁹.

The specific pathogen free (SPF) dogs needed to be matched with diseased dogs.

Venous blood needed to be regularly collected for hematology and serum biochemical testing before blood sampling of the matched dogs, and each index was within a normal range.

Peripheral blood was directly collected.

### Example 2

Greyhounds aged at 2-4 years old and having negative blood and a weight of 20-40 kg were selected.

The dogs were immunized with a conventional pentavaccine, an indirect ELISA method was used for testing, and the antibody titer thereof reaches over 2⁹.

The greyhounds needed to be matched with diseased dogs.

Venous blood needed to be regularly collected for hematology and serum biochemical testing before blood sampling of the matched dogs, and each index was within a normal range.

A mobilization agent (recombinant human granulocyte colony stimulating factor injection rhG-CSF) was injected every day before the hematopoietic stem cells were collected.

The injection amount of the mobilization agent was 150 μg/kg per day, and subcutaneous injection was performed once per day for five continuous days.

First time of hematopoietic stem cell collection was performed on the fifth day, and when the hematopoietic stem cells collected for the first time were not enough, second time of collection was performed.

### Example 3

Greyhounds aged at 2-4 years old and having negative blood and a weight of 20-40 kg were selected.

The dogs were immunized with a conventional pentavaccine, an indirect ELISA method was used for testing, and the antibody titer thereof reaches over 2⁹.

The SPF dogs needed to be matched with diseased dogs.

Venous blood needed to be regularly collected for hematology and serum biochemical testing before blood sampling of the matched dogs, and each index was within a normal range.

A mobilization agent (recombinant human granulocyte colony stimulating factor injection rhG-CSF) was injected every day before the hematopoietic stem cells were collected.

The injection amount of the mobilization agent was 150 μg/kg per day, and subcutaneous injection was performed once per day for five continuous days.

First time of hematopoietic stem cell collection was performed on the fifth day, and when the hematopoietic stem cells collected for the first time were not enough, second time of collection was performed.

### Example 4

SPF greyhounds aged at 2-4 years old and having negative blood and a weight of 20-40 kg were selected.

The dogs were immunized with a conventional pentavaccine, an indirect ELISA method was used for testing, and the antibody titer thereof reaches over 2⁹.

The SPF dogs needed to be matched with diseased dogs.

Venous blood needed to be regularly collected for hematology and serum biochemical testing before blood sampling of the matched dogs, and each index was within a normal range.

A mobilization agent (recombinant human granulocyte colony stimulating factor injection rhG-CSF) was injected every day before the hematopoietic stem cells were collected.

The injection amount of the mobilization agent was 300 μg/kg per day, and subcutaneous injection was performed once per day for five continuous days.

First time of hematopoietic stem cell collection was performed on the fifth day, and when the hematopoietic stem cells collected for the first time were not enough, second time of collection was performed.

### Example 5

Greyhounds aged at 2-4 years old and having negative blood and a weight of 20-40 kg were selected.

The dogs were immunized with a conventional pentavaccine, an indirect ELISA method was used for testing, and the antibody titer thereof reaches over 2⁹.

The SPF dogs needed to be matched with diseased dogs.

Venous blood needed to be regularly collected for hematology and serum biochemical testing before blood sampling of the matched dogs, and each index was within a normal range.

A mobilization agent (recombinant human granulocyte colony stimulating factor injection rhG-CSF) was injected every day before the hematopoietic stem cells were collected.

The injection amount of the mobilization agent was 150 μg/kg per day, and subcutaneous injection was performed once per day for five continuous days.

First time of hematopoietic stem cell collection was performed on the fifth day, and when the hematopoietic stem cells collected for the first time were not enough, second time of collection was performed.

The dogs were fed with royal jelly every day before the hematopoietic stem cells were collected.

### Data Processing and Analysis

Before the mobilization agent was injected, vein blood taking was performed on 20 Beagle dogs in a fasting state, and the white blood cells (WBC), hemoglobin (HGB), the red blood cells (RBC), hematocrit (HCT), a mean corpuscular volume (MCV), a mean corpuscular Hb content (MCH), a mean corpuscular Hb concentration (MCHC), and platelets (PLT) were measured by using a globulimeter. Serum was separated, and blood glucose (GLU), blood urea nitrogen (BUN), total bilirubin (T-BIL), total cholesterol (TCH), total protein (TP), albumin (ALB), aspartate aminotransferase (AST), alanine aminotransferase (ALT), alkaline phosphatase (ALP) and other biochemical indicators are measured by using a full-automatic biochemical analyzer.

TABLE 1
Blood routine values of Beagle dogs before
injection of mobilization agent

| Index | Male | Female |
|---|---|---|
| WBC/109 L⁻¹ | 11.57 ± 2.91 | 12.72 ± 2.46 |
| RBC/1012 L⁻¹ | 6.64 ± 1.94 | 7.23 ± 1.74 |
| HGB/g L⁻¹ | 152.84 ± 12.36 | 167.53 ± 27.69 |
| HCT/% | 18.14 ± 21.65 | 17.64 ± 21.37 |
| MCV/fL | 65.66 ± 12.27 | 58.73 ± 12.97 |
| MCH/pg | 22.34 ± 1.46 | 23.57 ± 2.62 |
| MCHC/g L⁻¹ | 358.25 ± 58.02 | 401.62 ± 54.06 |
| PLT/109 L⁻¹ | 278.46 ± 58.30 | 263.28 ± 60.24 |

TABLE 2
Blood biochemical values of Beagle dogs
before injection of mobilization agent

| Index | Male | Female |
|---|---|---|
| GLU/mmol L⁻¹ | 4.75 ± 0.54 | 4.75 ± 0.46 |
| CRE/umol L⁻¹ | 60.78 ± 7.95 | 93.85 ± 7.48 |
| BUN/mmol L⁻¹ | 1.95 ± 0.54 | 2.45 ± 0.86 |
| T-BIL/umol L⁻¹ | 7.69 ± 2.47 | 6.45 ± 2.95 |
| TCH/mmol L⁻¹ | 4.55 ± 0.96 | 4.59 ± 0.68 |
| TP/g L⁻¹ | 50.31 ± 4.13 | 45.31 ± 4.86 |
| ALB/g L⁻¹ | 27.52 ± 2.00 | 24.20 ± 4.23 |
| ALT/U L⁻¹ | 28.43 ± 10.25 | 31.25 ± 12.25 |
| AST/U L⁻¹ | 21.23 ± 5.05 | 21.10 ± 4.17 |
| ALT/U L⁻¹ | 155.56 ± 31.25 | 148.48 ± 45.03 |

It may be seen from the above that the blood routine and biochemical values of the Beagle dogs before injection of the mobilization agent all satisfy the requirements, and are suitable for collection of the hematopoietic stem cells.

In conclusion, the examples of the present disclosures provide a preparation method for canine whole blood hematopoietic stem cells. The experimental dogs are the SPF dogs free of special pathogens, have a large number of antibodies in blood, have great advantages in the aspects of safety performance and nutrition, and the content of the hematopoietic stem cells is greatly improved by injecting the mobilization agent and feeding the royal jelly, such that the hematopoietic stem cells prepared through the method not only have good safety performance, but also contain rich antibodies with a high content.

According to the present disclosure, the experimental dogs, namely the SPF dogs are free of special pathogens, and have great advantages in the aspect of safety performance, such that the prepared blood has good safety performance. The hematopoietic stem cells are provided for other diseased dogs by using the SPF greyhounds as volunteers, which not only improves the safety of blood transfusion, but also greatly improves a cure rate of the diseased dogs, and plays a crucial role in diagnosis and treatment of the diseased dogs.

What are described above are merely preferred particular embodiments of the present disclosure, but a protection scope of the present disclosure is not limited thereto. Equivalent substitutions or changes that are made by any of those skilled in the art and familiar with the technical field within the technical scope disclosed by the present disclosure on the basis of the technical solution and concepts of the present disclosure should be covered within the protection scope of the present disclosure.

What is claimed is:

1. A preparation method for canine whole blood hematopoietic stem cells, comprising the following steps:
step 1: performing a matching of dogs, wherein a blood type of specific pathogen free (SPF) dogs is a negative blood, immunizing the SPF dogs with a conventional pentavaccine, and using an indirect ELISA method for testing, wherein an antibody titer of each of the SPF dogs reaches over 2⁹, and matching the SPF dogs with diseased dogs;
step 2: performing a physical examination of the SPF dogs, regularly collecting a venous blood of the SPF dogs for a hematology and serum biochemical testing before a blood sampling of the SPF dogs, and each index of the hematology and serum biochemical testing is within a normal range; and
step 3: collecting the canine whole blood hematopoietic stem cells of the SPF dogs, injecting a mobilization agent in the SPF dogs every day before the canine whole blood hematopoietic stem cells are collected, and feeding the SPF dogs a royal jelly before the canine whole blood hematopoietic stem cells are collected to improve a content of the canine whole blood hematopoietic stem cells.

2. The preparation method for the canine whole blood hematopoietic stem cells according to claim 1, wherein step 1, step 2 and step 3 are performed in a complete sterile closed room.

3. The preparation method for the canine whole blood hematopoietic stem cells according to claim 1, wherein the dogs are the SPF dogs, and the SPF dogs comprise greyhounds, Beagle dogs, Dobermans, boxer dogs, and German shepherd dogs, and the SPF dogs have an age of 2-4 years old and a weight of 20-40 kg.

4. The preparation method for the canine whole blood hematopoietic stem cells according to claim 1, wherein the mobilization agent is a recombinant human granulocyte colony stimulating factor injection rhG-CSF.

5. The preparation method for the canine whole blood hematopoietic stem cells according to claim 1, wherein an injection amount of the mobilization agent is 150 μg/kg per day, and a subcutaneous injection of the mobilization agent is performed once per day for five continuous days.

6. The preparation method for the canine whole blood hematopoietic stem cells according to claim 5, wherein a first time collection of the canine whole blood hematopoietic stem cells is performed on a fifth day of the subcutaneous injection of the mobilization agent.

7. The preparation method for the canine whole blood hematopoietic stem cells according to claim 6, wherein when the first time collection of the canine whole blood hematopoietic stem cells is not enough, a second time of collection of the canine whole blood hematopoietic stem cells is performed.
